(19) United States
(12) Patent Application Publication
(10) Pub. No.: US 2026/0069124 A1
(43) Pub. Date: Mar. 12, 2026

(54) ILLUMINATION DEVICE FOR A MEDICAL IMAGING UNIT SUCH AS AN ENDOSCOPE, EXOSCOPE, AND/OR MICROSCOPE

(71) Applicant: KARL STORZ SE & Co. KG, Tuttlingen (DE)

(72) Inventor: Jan-Heiko TISCHLER, Tuttlingen (DE)

(21) Appl. No.: 19/318,113

(22) Filed: Sep. 3, 2025

(30) Foreign Application Priority Data
Sep. 6, 2024 (DE) 10 2024 125 695.8

(51) Int. Cl. A61B 1/06

(52) U.S. Cl. CPC A61B 1/0684; A61B 1/0638; A61B 1/0646

(57) ABSTRACT

An illumination device (10), in particular for a medical imaging unit (11) such as an endoscope, exoscope, and/or microscope, comprising:

an illumination unit (14) that is configured to provide illumination with an illumination spectrum (54),

a further illumination unit (60) that is configured to provide further illumination with a further illumination spectrum (56), and

a combination unit (65) that is configured to combine at least a portion of the illumination and at least a portion of the further illumination to form a combination illumination, wherein the combination unit (65) has a beam splitter element (66) that has a first region (40) that is substantially reflective for the illumination and a second region (42) that is substantially transmissive for the further illumination, which second region is arranged next to the first region (40) and is preferably at least partially surrounded by the first region (40).

### CROSS-REFERENCE TO RELATED APPLICATIONS

This Application claims the benefit Under 35 U.S.C. 119(a) to German Patent Application No. 10 2024 125 695.8, filed 6 Sep. 2024, the disclosure of which is incorporated herein by reference in its entirety

### SUMMARY

The present invention relates to an illumination device for a medical imaging unit such as an endoscope, exoscope, and/or microscope, a medical, in particular endoscopic, exoscopic, and/or microscopic, imaging device with such an illumination device, and a method for operating an illumination device.

Imaging devices, such as endoscopic or exoscopic devices that produce multispectral or hyperspectral images, are known from the prior art. Multispectral or hyperspectral images have a spectral dimension in addition to two spatial dimensions, such as a conventional image from a camera. The spectral dimension includes multiple spectral bands (wavelength bands). Multi-spectral and hyperspectral images differ substantially in the number and width of their spectral bands.

Some imaging devices are known for producing such multispectral or hyperspectral images, especially in the context of medical applications. For example, DE 20 2014 010 558 U1 describes a device for recording a hyperspectral image of a region under examination of a body. The device includes an input lens for generating an image in an image plane and a slit-shaped aperture in the image plane for masking out a slit-shaped region of the image. The light passing through the aperture is spread out by a dispersive element and recorded by a camera sensor. This allows the camera sensor to record a plurality of spectra, each with an associated spatial coordinate, along the longitudinal direction of the slit-shaped aperture. The device described is further configured to record further spectra along the longitudinal direction of the slit-shaped aperture in a direction different from the longitudinal direction of the slit-shaped aperture. The method underlying this disclosure for generating multispectral or hyperspectral images is also known as the so-called pushbroom method.

In addition to the pushbroom method, there are other methods for generating multispectral or hyperspectral images. In the so-called whiskbroom method, the region under study or else the object is scanned point-by-point, and a spectrum is obtained for each point. In contrast, the staring method involves taking multiple images with the same spatial coordinates. Various spectral filters and/or illumination sources are used from image to image to resolve spectral information. Furthermore, there are methods according to which a two-dimensional, multi-color image is broken down into several individual spectral images using suitable optical elements, such as optical slicers, lenses, and prisms, which images are simultaneously acquired on different detectors or detector regions. This is sometimes referred to as the snapshot approach.

As described in DE 10 2020 105 458 A1, multispectral and hyperspectral imaging devices are particularly suitable as endoscopic imaging devices. In this connection, multispectral and/or hyperspectral imaging is a fundamental field of application—for example, for diagnostics and for assessing the success or quality of an intervention.

In addition, white-light imaging is used in particular in the medical imaging field. Observed tissue is illuminated with white light, and images of the tissue are generated using a camera or other image acquisition sensor system, which can then be displayed to a user.

Fluorescence imaging is also used, in particular in the medical imaging field. Tissue is illuminated in a certain wavelength range in order to excite fluorescent dye molecules that have been specifically introduced into certain entities such as tissue regions. The subsequently emitted light with a longer wavelength can be observed through a suitably selected filter, by means of which the excitation light can be blocked.

Multimodal imaging devices allow the capture of, optionally, white-light images and/or multi-spectral images and/or fluorescence images and/or hyperspectral images. Examples of such imaging devices are multimodal endoscopes and multimodal exoscopes. To realize different modes, illumination devices can be required that are operable in different illumination modes in order to generate illumination light in different spectral ranges, as needed.

From U.S. Pat. Nos. 10,481,095 B2 and 11,668,922 B2, illumination devices with several illumination sources are known, the light emitted by each of which can be combined by means of beam splitter elements.

Based upon the prior art, the invention is based in particular upon but not limited to the object of providing reliable and efficient illumination of a region under examination.

This object is achieved according to the invention by an illumination device, an endoscopic, exoscopic, and/or microscopic imaging unit, and a method for operating an illumination device as described herein and defined in the claims.

The invention is based upon an illumination device for a medical imaging unit such as an endoscope, exoscope, and/or microscope, comprising:
an illumination unit that is configured to provide illumination with an illumination spectrum,
a further illumination unit which is configured to provide further illumination with a further illumination spectrum, and
a combination unit that is configured to combine at least a portion of the illumination and at least a portion of the further illumination to form a combination illumination.

In one aspect of the invention, the combination unit has a beam splitter element that has a first region that is substantially reflective for the illumination and a second region that is substantially transmissive for the further illumination, which second region is arranged next to the first region and is preferably at least partially and particularly preferably completely surrounded by the first region.

In a further aspect of the invention, which can in particular also be considered independently of the previously mentioned aspect, the combination unit is configured to combine the illumination spectrum and the further illumination spectrum in each case substantially unchanged.

The invention further relates to a medical imaging unit, in particular an endoscopic, exoscopic, and/or microscopic medical imaging unit, with the aforementioned illumination device.

The invention further relates to a method for operating an illumination device, in particular for a medical imaging unit such as an endoscope, exoscope, and/or microscope, wherein the illumination device comprises:
an illumination unit that is configured to provide illumination with an illumination spectrum, and
a further illumination unit that is configured to provide further illumination with a further illumination spectrum,

wherein at least a portion of the illumination and at least a portion of the further illumination are combined to form a combination illumination.

In one aspect of the invention, the portion of the illumination and the portion of the further illumination are combined to form the combination illumination by means of a beam splitter element that has a first region that is substantially reflective for the illumination and a second region that is substantially transmissive for the further illumination, which second region is arranged next to the first region and is preferably at least partially and particularly preferably completely surrounded by the first region.

In a further aspect of the invention, which can in particular also be considered independently of the previously mentioned aspect, the illumination spectrum and the further illumination spectrum are in each case combined substantially unchanged.

The features according to the invention can provide a reliable and efficient illumination of a region under examination, especially if radiation power is to be increased in certain wavelength ranges—for example, for fluorescence excitation. In particular, a spectral change, i.e., in particular a spectral fragmentation, as can occur, for example, when using conventional beam splitters with dichroic filters, can advantageously be avoided even with overlapping illumination spectra. This can lead to a more natural color impression of an imaged region under examination. In addition, radiation losses and/or scattered light can be minimized during the combining. Compared to dichroic filters, costs and/or tolerance requirements can be advantageously reduced, in particular when using laser light. In particular, advantageous operation in different modes can be made possible. In this process, a high degree of efficiency and/or operating safety and simplicity can be achieved. The proposed combination of imaging modes or illumination sources used for this purpose can reduce the complexity of light source and/or image acquisition. A small number of installed illumination sources, filters, and/or associated optical elements can be achieved, while at the same time maintaining a wide range of functions. Furthermore, installation space can be saved, which allows a high degree of compactness to be achieved.

The first region and the second region are spatial regions of the beam splitter element of the combination unit. The two spatial regions differ structurally from one another. For example, the first region can comprise a material or be formed from a material or material composition that differs from a material or material composition of the second region. In addition, the second region could be formed at least partially and preferably completely by a material recess. The first region can be substantially opaque, particularly preferably substantially reflective, to further illumination. The second region can be substantially transmissive for the illumination.

“Substantially” opaque, reflective, or transmissive can be understood to mean that at least 90%, preferably at least 95%, and particularly preferably at least 98%, of a radiation intensity is not let through, reflected, or transmitted.

The fact that the combination unit is configured to combine the illumination spectrum and the further illumination spectrum “substantially unchanged” in each case can be understood in the present case to mean that the combination unit, in order to form the combination illumination, either maintains an intensity for all wavelengths of the illumination spectrum or reduces it proportionally for all wavelengths by the same proportionality factor, wherein the same can apply to the further illumination spectrum. In particular, due to the combination unit, a fragmentation of the illumination spectrum and the wider illumination spectrum does not take place. In particular, no wavelength ranges of the illumination spectrum and/or the wider illumination spectrum are filtered out.

The illumination device can be part, in particular a subassembly, of a medical, in particular endoscopic, exoscopic, and/or microscopic, imaging device. The imaging device can have a medical imaging unit such as an endoscope, exoscope, and/or microscope, which can be supplied with output illumination via a light guide, which can also be part of the imaging device. The output illumination can be formed at least partially, preferably to a large extent and particularly preferably substantially, by the combination illumination. The imaging unit can be configured to provide at least a portion, preferably at least a majority, and particularly preferably substantially all, of the output illumination and/or combination illumination for lighting and/or illuminating a region under examination. The imaging device and in particular the illumination device can be configured to provide light in a wavelength range of 100 nm to 1 mm, preferably 200 nm to 1,000 nm and particularly preferably 400 nm to 1,000 nm. in the form of a broad, continuous spectrum and/or in the form of several separate, partial spectra.

In some embodiments, the imaging device and, in particular, the imaging unit is configured to be insertable into a cavity for inspection and/or observation, e.g., into an artificial and/or natural cavity, such as into the interior of a body, into a body organ, into tissue, or the like. The imaging device and, in particular, the imaging unit can also be configured to be insertable into a housing, casing, shaft, tube, or other, in particular artificial, structure for inspection and/or observation.

The imaging device and, in particular, the imaging unit can be configured to acquire tissue parameters, images of wounds, images of body parts, etc. For example, the imaging device can be configured to image a surgical field. The imaging device and/or the imaging unit can comprise a spatially and spectrally resolving image acquisition unit which comprises at least one optical system and at least one image acquisition sensor system coupled to the optical system, which are configured to carry out image acquisition in which spatially and spectrally resolved image data are generated that comprise both spatial and spectral information.

The image acquisition unit and, in particular, the optical system and/or the image acquisition sensor system can be configured for multispectral and/or hyperspectral imaging, in particular for acquiring and/or generating multispectral and/or hyperspectral image data. Multispectral imaging or multispectral image data can refer in particular to such imaging, in which at least two, in particular at least three, and in some cases at least five, spectral bands can be acquired and/or are acquired independently of one another. Hyperspectral imaging or hyperspectral image data can refer in particular to imaging in which at least 20, at least 50, or even at least 100, spectral bands can be acquired and/or are acquired independently of one another.

In some embodiments, the imaging device and/or the imaging unit comprises a white-light camera and/or sensor system for white-light imaging. The imaging device and/or the imaging unit can be configured for white-light imaging in addition to spectrally resolved imaging. A separate optical system and/or a common optical system can be used for this purpose. White-light imaging and spectrally resolved imaging can be performed simultaneously or alternately, or sometimes simultaneously and sometimes sequentially.

In some embodiments, the imaging device and/or the imaging unit comprises a sensor system for fluorescence imaging. The imaging device and/or the imaging unit can be configured for fluorescence imaging in addition to spectrally resolved imaging and, if applicable, in addition to white-light imaging. A separate optical system and/or a common optical system can be used for this purpose. Fluorescence imaging, white-light imaging (if applicable), and spectrally resolved imaging can be performed simultaneously or alternately, or sometimes simultaneously and sometimes sequentially.

For some applications, it can be advantageous to be able to use a high spectral resolution. Hyperspectral imaging is then recommended. It can be combined with white-light imaging and/or fluorescence imaging. This makes real-time observation possible via a white-light image and/or a fluorescence image, even if the acquisition of spectrally resolved image data only occurs substantially in real time, i.e., for example, several seconds are needed to create a spectrally resolved image.

For some applications, it can be advantageous to generate spectral image data in real time. This includes, for example, the generation of a spectrally resolved image in less than a second or even several times per second. It can be useful to use multispectral imaging in this case. An optionally lower spectral resolution is then offset by a higher refresh rate. Depending upon the application, it can be sufficient to consider only a few different spectral ranges and/or wavelengths—for example, two or three or four, or generally less than ten. In this case, additional white-light imaging can optionally be omitted. Spectrally resolved image data that are acquired in real time or provide several images per second can also be used for monitoring purposes, wherein it is not absolutely necessary to create a reproducible image for a user, but, rather, the image data can also be processed in the background.

The optical interface can be fixed or optionally detachable or connectable. In addition, the optical interface can be combined with a mechanical interface so that an optical connection is automatically established—for example, when the imaging unit is mechanically coupled.

The imaging device can have a control system that is configured to automatically match the operating state of the imaging unit and the illumination mode of the illumination device, in particular the illumination unit. The controller can be configured to control the illumination device and/or the imaging unit—for example, for MSI or PDD applications.

The illumination unit can have one or more illumination sources—for example, a first illumination source and a second illumination source and in particular also a third illumination source. The illumination unit is configured to provide optical radiation, in particular in the wavelength range of 100 nm to 1 mm, preferably in the wavelength range of 200 nm to 1,000 nm, and can comprise infrared radiation, visible light, and/or UV radiation. By means of the optical radiation, a region under examination can be lit, e.g., with white light, and/or excitation radiation can be provided to excite a luminescent dye, preferably a fluorescent dye, such as cyanine 5.5 (Cy 5.5), indocyanine green (ICG), pafolacianin (OTL 38), VisBlue, ViRed, NIR1, or fluorescein.

Individual illumination sources of the illumination unit can be designed to emit optical radiation of a certain wavelength range and/or a certain radiation spectrum. The illumination sources are preferably all designed to emit optical radiation in different wavelength ranges and/or with different radiation spectra, wherein overlaps of the radiation spectra would be conceivable. For example, at least one of the illumination sources could be designed to emit red light, in particular in the wavelength range of 640 nm to 780 nm, at least one of the illumination sources could be designed to emit green light, in particular in the wavelength range of 490 nm to 570 nm, and/or at least one of the illumination sources could be designed to emit blue light, in particular in the wavelength range of 430 nm to 490 nm. Furthermore, at least one of the illumination sources could be designed to emit infrared radiation, in particular in the wavelength range of 780 nm to 1 mm, and/or at least one of the illumination sources could be designed to emit UV radiation, in particular in the wavelength range of 100 nm to 380 nm. Furthermore, one of the illumination sources could be designed to emit visible light, in particular in the wavelength range of 380 to 780 nm.

The illumination sources can be designed as any light emitters that appear appropriate to a person skilled in the art—for example, as gas discharge lamps, in particular xenon gas discharge lamps. In a preferred embodiment, at least one illumination source of the illumination unit comprises an LED or is designed as such. Particularly preferably, all illumination sources of the illumination unit in each case comprise an LED or are designed as such. As a result, advantageous illumination is provided, which is particularly cost-effective and/or easy to control electronically.

Preferably, the first illumination source is configured to emit a continuous LED light spectrum having an intensity maximum at 940 nm. Preferably, the second illumination source is configured to emit a continuous LED light spectrum having an intensity maximum at 660 nm. Preferably, the third illumination source is configured to emit a continuous LED light spectrum having intensity maxima at 440 nm and 550 nm.

In addition, the illumination unit can have at least one optical element for beam shaping, beam splitting and/or beam combining, and/or beam deflection. The optical element can be configured to shape radiation provided by one or more of the illumination sources, in particular to bundle an optical beam, and/or to split radiation provided by one or more of the illumination sources into two beam paths or to combine two beams provided by at least two of the illumination sources, and/or to redirect radiation provided by one or more of the illumination sources, in particular to thereby provide the illumination of the illumination unit.

The further illumination unit can have one or more further illumination sources—for example, a further first illumination source and a further second illumination source and in particular also a further third illumination source. The further illumination unit is configured to provide further optical radiation, in particular in the wavelength range of 100 nm to 1 mm, preferably in the wavelength range of 400 nm to 800 nm, and can comprise infrared radiation, visible light, and UV radiation. By means of the further optical radiation, a region under examination can be lit, preferably with white light, and/or excitation radiation can be provided to excite a luminescent dye, preferably a fluorescent dye, such as cyanine 5.5 (Cy 5.5), indocyanine green (ICG), pafolacianin (OTL 38), VisBlue, ViRed, NIR1, or fluorescein.

Individual further illumination sources of the further illumination unit can be designed to emit further optical radiation of a certain wavelength range and/or a certain radiation spectrum. The further illumination sources are preferably all designed to emit further optical radiation at different wavelength ranges and/or with different radiation spectra, wherein overlaps are conceivable. For example, at least one of the further illumination sources could be designed to emit red light, in particular in the wavelength range of 640 nm to 780 nm, at least one of the further illumination sources could be designed to emit green light, in particular in the wavelength range of 490 nm to 570 nm, and/or at least one of the further illumination sources could be designed to emit blue light, in particular in the wavelength range of 430 nm to 490 nm. Furthermore, at least one of the further illumination sources could be designed to emit infrared radiation, in particular in the wavelength range of 780 nm to 1 mm, and/or at least one of the further illumination sources could be designed to emit UV radiation, in particular in the wavelength range of 100 nm to 380 nm.

The illumination sources can be designed as any light emitters that appear appropriate to a person skilled in the art—for example, as gas discharge lamps or as LED's. In a preferred embodiment, at least one further illumination source of the further illumination unit comprises a laser or a laser diode or is designed as such. Particularly preferably, all further illumination sources of the further illumination unit in each case comprise a laser or a laser diode or are designed as such. As a result, advantageous narrow-band illumination can be provided. Furthermore, a radiation power density can be achieved.

Preferably, the further first illumination source is configured to emit a Gaussian beam and/or a laser line at a wavelength of 470 nm. Preferably, the further second illumination source is configured to emit a Gaussian beam and/or a laser line at a wavelength of 640 nm. Preferably, the further third illumination source is configured to emit a Gaussian beam and/or a laser line at a wavelength of 770 nm. Furthermore, it would be conceivable for the further illumination unit to have only one further illumination source and for this to be configured to emit a Gaussian beam and/or a laser line at a wavelength of 470 nm, 640 nm, or 770 nm. However, the further illumination sources mentioned can also be configured to emit other wavelengths.

The further illumination unit can have at least one further optical element for beam shaping, beam splitting and/or beam combining, and/or beam deflection. The further optical element can be configured to shape radiation provided by one or more of the further illumination sources, in particular to bundle an optical beam, and/or to split radiation provided by one or more of the further illumination sources into two beam paths and/or to combine two beams provided by at least two of the further illumination sources, and/or to redirect radiation provided by one or more of the further illumination sources, in particular to thereby provide the further illumination of the further illumination unit.

The illumination sources and/or the further illumination sources can be activated independently of one another or in groups and, in particular, also only temporarily and/or sequentially. The illumination unit and/or the further illumination unit can be operable in at least one multispectral mode in which a first group of the illumination sources and/or the further illumination sources is at least temporarily activated, and in which the illumination unit and/or the further illumination unit provides illumination light for multispectral imaging. Furthermore, the illumination unit and/or the further illumination unit can be operable in at least one fluorescence mode in which a second group of the illumination sources and/or the further illumination sources is at least temporarily activated, and in which the illumination unit and/or the further illumination unit provides illumination light for fluorescence imaging. The illumination sources and/or the further illumination sources can comprise at least one illumination source that is included in both the first group and the second group. It is understood that mixed operating modes can also occur, in which said modes are used sequentially. For example, multispectral imaging and fluorescence imaging can be performed sequentially.

In some embodiments, the illumination spectrum in the wavelength space overlaps at least partially with the wider illumination spectrum. As a result, the radiation power in the overlapping range of the wavelength space can be advantageously adapted and, in particular, increased.

The illumination spectrum can also be formed by individual illumination spectra, which are separated from one another in particular in the wavelength space, and which can originate in particular from individual illumination sources of the illumination unit. One of these individual illumination spectra can overlap with the further illumination spectrum, which in turn can be formed by individual further illumination spectra, in particular those separated from one another in the wavelength space, which can originate from further illumination sources of the further illumination unit.

The illumination spectrum can be broader-band than the wider illumination spectrum, whereby advantageous application possibilities can be made possible. In particular, the illumination spectrum can be used to light the region under examination. However, the wider illumination spectrum can advantageously support and/or make possible fluorescence excitation.

Here, “broader-band” can also refer to the fact that individual illumination spectra generated by illumination sources of the illumination unit are broader-band than further individual illumination spectra generated by further illumination sources of the further illumination unit.

The illumination device can comprise an illumination output that can have an optical interface, in particular for connection to the light guide, and can be configured to provide the output illumination.

The illumination device can comprise a first optical path from the first illumination source to the illumination output and at least one second optical path from the second illumination source to the illumination output.

An “optical path” can be understood in the present case as a unit that is defined by optical elements, e.g., for beam shaping, for beam splitting and/or combining and/or for beam deflection, and optical path lengths along a beam path, and that extends along the beam path between one of the illumination sources and the illumination output. The optical elements can be part of the corresponding optical path. The first optical path and the second optical path can partially overlap, so that an optical element of the first optical path can also be part of the second optical path.

The first optical path and the second optical path can in each case have exactly three optical beam-shaping elements, preferably lenses.

An “optical beam-shaping element” can be understood as an optical element that is configured for beam shaping, in particular for beam bundling or beam expansion. Diffractive optical elements, achromats, and/or single lenses, particularly preferably converging lenses, can be used as optical beam-shaping elements. In the case of optical beam-shaping elements that are based upon the interaction of several optical sub-elements, e.g., several lenses of an achromat, these sub-elements are not to be counted individually as optical beam-shaping elements, but only together as an individual optical beam-shaping element. Beam splitter elements are not to be understood as optical beam-shaping elements.

Particularly preferably, the exactly three optical beam-shaping elements are designed as a condenser lens or a condenser, a compensator lens, in particular a converging lens, and as a focus lens. Condenser lenses or condensers can generally be configured to reduce a divergence of a particular associated illumination source and/or to introduce as large a portion as possible of a radiation provided by the associated illumination source into an imaging beam path and/or to at least partially collimate the provided radiation. Condenser lenses or condensers can generally provide uniform lighting. Compensator lenses can generally be configured to compensate for and narrow an expanding beam path, in particular to collimate or focus it. If applicable, several lenses, in particular convex lenses, which interact in a condenser lens or a condenser are not to be counted individually; rather, the combination of these lenses in the form of a condenser lens or a condenser represents an individual optical beam-shaping element. The same applies to a compensator lens.

In some embodiments, all optical paths from illumination sources of the illumination unit to the illumination output can in each case have at most and preferably exactly three optical beam-shaping elements, preferably lenses. As a result, a small number of installed elements can be achieved, while maintaining a wide range of functions. Furthermore, installation space can be saved, which allows a high degree of compactness to be achieved. By using a maximum of three optical beam-shaping elements in the optical paths, it is particularly advantageous to make possible an alignment of imaging properties of different illumination sources.

In a further embodiment, the first optical path and/or the second optical path can have at least two beam splitter elements arranged optically directly one behind the other, whereby an optical design can be advantageously simplified. In particular, the alignment of imaging properties with respect to different illumination sources can be simplified. In some embodiments, a beam splitter element can be designed as a cross beam splitter, which comprises two individual beam splitters arranged at an angle, preferably perpendicularly, to one another. One or both of the beam splitter elements can be configured to combine at least two beam paths and preferably direct them in the direction of the illumination output. One of the beams can be transmitted through the beam splitter element, while the other beam can be reflected by the beam splitter element. Transmission and reflection properties of the beam splitter element can preferably be matched to the spectra of the two beams in order to advantageously avoid radiation losses. The beam splitter elements and individual beam splitters described herein can be designed as any optical elements that appear appropriate to a person skilled in the art, preferably having at least one semi-transparent mirror and/or at least one interference mirror or filter, preferably at least one dichroic mirror and/or filter.

The beam splitter elements or individual beam splitters described herein can in each case be designed as any optical element that appears appropriate to a person skilled in the art, but preferably as a semi-transparent mirror and/or an interference mirror or filter, preferably as a dichroic mirror and/or filter. The beam splitter element can be configured to combine two beam paths and direct them in the direction of the illumination output. One of the beams is transmitted through the beam splitter element; the other beam is reflected by the beam splitter element. Transmission and reflection properties of the beam splitter element can preferably be matched to the spectra of the two beams in order to advantageously avoid radiation losses.

The beam splitter elements or individual beam splitters described herein can comprise band-pass filters, in particular notch filters, such that in each case they exhibit a high reflectance and a low transmittance in a narrow spectral band or in narrow spectral bands, but otherwise a high transmittance and a low reflectance, or vice versa. Alternatively or additionally, the beam splitter elements or individual beam splitters can comprise edge filters, such that they exhibit a high reflectance and a low transmittance in a spectral band up to an edge, but otherwise a high transmittance and a low reflectance, or vice versa. The spectral position and/or width of the corresponding notch and/or the spectral position of the edge can be adapted to the spectral range of the particular associated illumination source or sources, such that its or their light can be largely deflected, but light from other illumination sources can be largely transmitted.

The beam splitter elements can be arranged in an optical beam path starting from the illumination sources, both in front of or both behind the compensator lens. It would also be conceivable that one of the beam splitter elements be arranged in front of the compensator lens, and the other beam splitter element be arranged behind the compensator lens. As a whole, the first optical path and/or the second optical path could comprise three beam splitter elements. One of the beam splitter elements could be designed as the beam splitter element of the combination unit.

Advantageously, the first optical path and the second optical path are of the same length, whereby deviations and, if applicable, measurement errors can be avoided, which are due to relative spectral intensities in different spectral ranges and can occur, for example, if an endoscope (shaft) is rotated relative to a camera unit and/or if a light guide is rotated relative to the imaging unit. Due to the substantially equal length of the light paths, largely identical intensity profiles of the affected illumination sources can be achieved. The term “equal length” is to be understood here and below to mean that two lengths are equal in the context of tolerances, in particular in the context of manufacturing and assembly tolerances, and/or that a relative deviation of the two lengths is a maximum of 1%, preferably a maximum of 0.1%, and particularly preferably a maximum of 0.01%.

In some embodiments, the first optical path and the second optical path can have the same optical beam-shaping elements, preferably lenses. As a result, identical imaging properties and/or light intensities can be made possible for the different optical paths. The first optical path and the second optical path can also partially have the same optical beamforming elements that are part of both the first and the second optical paths. Preferably, the first optical path and the second optical path have condenser lenses or condensers of the same type.

In some embodiments, the first optical path and the second optical path can be identical to one another. “Identical” optical paths can be understood to mean optical paths that are of equal length and that have the same, possibly partially the same, in particular jointly used, optical elements. As a result, identical imaging properties for both optical paths can be ensured, whereby in particular imaging quality can be increased particularly advantageously.

In a further development, the first optical path preferably has a first condenser lens or a first condenser, the second optical path preferably has a second condenser lens or a second condenser, and the first optical path and the second optical path preferably have a common compensator lens. As a result, a design can be advantageously simplified. By using certain optical elements together, tolerance influences can also be minimized, whereby it is particularly advantageous to achieve largely identical imaging properties for different illumination sources. Preferably, the first condenser lens is of the same type as the second condenser lens, or the first condenser is of the same type as the second condenser. The first optical path and the second optical path can have a common focus lens, wherein the common focus lens can be arranged in front of the illumination output. Viewed in the beam direction, the common compensator lens can be arranged between the first condenser lens or the first condenser, or the second condenser lens or the second condenser, and the common focus lens.

In a preferred embodiment, the illumination device can have a third optical path from a third illumination source of the illumination unit to the illumination output, wherein the third optical path can have a third condenser lens or a third condenser and a dedicated compensator lens or the common compensator lens. As a result, flexibility, in particular with regard to the use of installation space, can be advantageously increased. Furthermore, the need for installation space can be reduced if applicable, in particular by correspondingly deflecting a beam path. The third optical path may partially overlap with the first optical path and/or the second optical path, such that an optical element of the third optical path can also be part of the first optical path and/or the second optical path. The third condenser lens can be of the same type or a different type as the first and/or second condenser lens. Accordingly, the third condenser can be of the same type or different type as the first and/or second condenser. The third optical path and the first optical path and/or the second optical path can have a common focus lens, in particular the aforementioned common focus lens, wherein the common focus lens can be arranged in front of the illumination output. Viewed in the beam direction, the dedicated compensator lens can be arranged between the third condenser lens or the third condenser and the common focus lens.

In some embodiments, the first optical path, the second optical path, and the third optical path, and particularly preferably all optical paths from illumination sources of the illumination unit to the illumination output, can be identical to one another. As a result, identical imaging properties can be ensured for the three optical paths and particularly preferably for all optical paths from illumination sources of the illumination unit to the illumination output, whereby in particular imaging quality can be particularly advantageously increased.

In some embodiments, the first illumination source, the second illumination source, and/or the third illumination source can be configured for simultaneous illumination, in particular white-light illumination, and/or joint evaluation. As a result, imaging and evaluation quality can be particularly advantageously increased. The controller can be configured to operate the first illumination source, the second illumination source, and/or the third illumination source simultaneously in at least one operating mode, and/or to use spectral signals from a wavelength range of the first illumination source, the second illumination source, and/or the third illumination source simultaneously in the context of an analysis and/or calculation.

The illumination device can additionally comprise a further first optical path from the further first illumination source to the illumination output. The beam splitter element of the combination unit can be the last beam splitter element in the beam direction in front of the illumination output.

In some embodiments, the further first optical path can have exactly two or exactly three optical beam-shaping elements, preferably lenses, whereby a small number of installed optical elements can be achieved, while at the same time maintaining a wide range of functions. Furthermore, installation space can be saved, which allows a high degree of compactness to be achieved. By using exactly two or exactly three optical beam-shaping elements in the optical path, it is particularly advantageously possible to make possible an alignment of the imaging properties of different illumination sources. Particularly preferably, the exactly two or exactly three optical beam-shaping elements are designed as a condenser lens or a condenser and a focus lens and possibly additionally a compensator lens, in particular a converging lens. The focus lens can be designed as the aforementioned common focus lens.

In further embodiments, the illumination device can comprise a further second optical path from the further second illumination source to the illumination output, wherein the further second optical path can have exactly two or exactly three optical beam-shaping elements, preferably lenses. As a result, a small number of installed optical elements can be achieved, while at the same time achieving a wide range of functions. Furthermore, installation space can be saved, which allows a high degree of compactness to be achieved. By using exactly two or exactly three optical beam-shaping elements in the optical path, it is particularly advantageously possible to make possible an alignment of the imaging properties of different illumination sources. Particularly preferably, the exactly two or exactly three optical beam-shaping elements are designed as a condenser lens or a condenser and a focus lens and possibly additionally a compensator lens, in particular a converging lens. The focus lens can be designed as the previously mentioned common focus lens.

The further first optical path and the further second optical path can be of the same length, whereby deviations and, if applicable, measurement errors can be avoided, which are due to relative spectral intensities in different spectral ranges and can occur, for example, if an endoscope (shaft) is rotated relative to a camera unit, and/or if a light guide is rotated relative to the imaging unit. Due to the substantially equal length of the light paths, largely identical intensity profiles of the affected further illumination sources can be achieved.

In some embodiments, the further first optical path and the further second optical path can have the same optical beam-shaping elements, preferably lenses. As a result, identical imaging properties can be achieved for the various further optical paths. The further first optical path and the further second optical path can also partially have the same optical beamforming elements that are part of both the further first and the further second optical paths. Preferably, the further first optical path and the further second optical path have condenser lenses or condensers of the same type.

In certain embodiments, an optical path from the particular (further) illumination source to the illumination output can be identical for all (further) illumination sources of the illumination unit and the further illumination unit. As a result, identical imaging properties can be ensured for all optical paths of the illumination device, whereby in particular imaging and illumination quality can be increased particularly advantageously.

The first optical path and/or the second optical path and/or the further first optical path and/or the further second optical path can have at least one beam deflection element for a pure deflection of a beam path. As a result, flexibility with regard to a design, in particular with respect to the arrangement of components, the use of installation space, and/or cooling, can be advantageously increased. The beam deflection element can be designed as any optical element that appears appropriate to a person skilled in the art—for example, as a light guide and/or preferably as a mirror.

Furthermore, the illumination device can comprise a detector unit for determining at least one illumination property of the output illumination and/or the combination illumination. As a result, imaging, illumination, and/or evaluation quality can be advantageously increased. In particular, thermal influences on the illumination spectrum or the illumination spectra due to heating and/or aging of illumination sources and/or optical elements can be taken into account and/or compensated for. As a result, a white balance can be improved, and/or an initial white balance can be corrected. The detector unit can have at least any optical detector that appears appropriate to a person skilled in the art—for example, a photodiode, a phototransistor, a photoresistor, a CCD sensor, a CMOS sensor, and/or a camera. The illumination device can be connected to the imaging unit, in particular the image acquisition unit, whereby feedback from the imaging unit can be provided to the illumination device, in particular for white balance.

The illumination property could be an illumination spectrum, an illumination intensity, and/or an illumination power. Preferably, the illumination property is a total radiant power of the output illumination and/or combination illumination, whereby an imaging, illumination, and/or evaluation quality can be increased particularly easily, wherein thermal influences can be taken into account and/or compensated for in a simple manner. In particular, white balance can be improved and/or corrected particularly easily.

In some embodiments, the second region for the illumination can be at least partially, and, in certain embodiments, substantially, transmissive, whereby detection and/or monitoring of a radiation power and/or composition of the illumination and/or the combination illumination and/or the output illumination can advantageously be made possible.

For this purpose, the detector unit can be used, which is configured to detect a proportion of the illumination that has passed through the second region. In this respect, the detector unit can be arranged on a side, facing away from the illumination unit, of the beam splitter element of the combination unit.

If the second region for the further illumination is at least partially reflective, detection and/or monitoring of a radiation power and/or composition of the further illumination and/or the combination illumination and/or the output illumination can advantageously be made possible.

A proportion of the further illumination that is reflected at the second region can constitute less than 10%, preferably less than 5%, and particularly preferably less than 2%, of a radiation intensity of the further illumination.

For this purpose, the detector unit can be used, which is configured to detect the proportion of the further illumination that was reflected at the second region. In this respect, the detector unit can be arranged on a side, facing the further illumination unit, of the beam splitter element of the combination unit.

In some embodiments, the beam splitter element of the combination unit can comprise a broadband mirror having an integrating pinhole aperture that defines the second region. As a result, an advantageously simple combination unit can be provided that, in particular, can combine the illumination spectrum and the further illumination spectrum in each case substantially unchanged.

In this case, the second region is defined by the region of the pinhole aperture. The pinhole aperture can exhibit a diameter of at least 1 mm, in particular at least 2 mm, and of at most 5 mm, in particular at least 4 mm. It can be arranged at any point on the broadband mirror. The broadband mirror can be designed to be flat or curved, in particular parabolic.

An orientation of a boundary wall or boundary walls of the pinhole aperture can be perpendicular or angular to a main surface of the beam splitter element of the combination unit, in particular of the broadband mirror, whereby the extent to which the illumination and/or the further illumination can pass through the second region is in particular adjustable. Thus, a hole for producing the pinhole aperture can be drilled perpendicularly to the main surface or at an angle of less than 90° to the main surface.

The beam splitter element of the combination unit can have a filling element that at least partially and preferably completely fills the pinhole aperture. The filling element could, for example, be a transparent window element. The beam splitter element of the combination unit can have an optical filter element in the second region, whereby the transmission and reflection properties of the second region can be advantageously changed and/or adjusted.

For example, the optical filter element could be partially transparent over a wavelength range of the illumination and/or the further illumination and reflect a small proportion, in particular less than 10%, preferably less than 5% and particularly preferably less than 2%, of an incident light intensity, and otherwise substantially transmit the light.

In addition, the optical filter element for the illumination could be substantially reflective and at least partially transmissive, wherein a proportion of the illumination transmitted at the optical filter element can represent less than 10%, preferably less than 5%, and particularly preferably less than 2%, of a radiation intensity of the illumination. Furthermore, the optical filter element for the further illumination could be substantially transmissive and at least partially reflective, wherein a proportion of the further illumination that is reflected at the optical filter element can amount to less than 10%, preferably less than 5%, and particularly preferably less than 2%, of a radiation intensity of the further illumination. In particular, the optical filter element could have a dedicated notch for each further illumination source of the further illumination unit.

In some embodiments, the beam splitter element of the combination unit can have a substrate that is substantially transmissive for the illumination and the further illumination, and a coating on the substrate that reflects broadband in the first region and that is at least partially and preferably substantially transmissive in the second region for the further illumination. As a result, an advantageously simple production can be made possible, whereby in particular costs can be reduced. Furthermore, a high degree of reliability can be achieved.

In particular, the coating can have a material recess in the second region. Alternatively, the coating in the second region for illumination can be designed to be at least partially, and preferably substantially, reflective.

The coating can be formed in two parts, with a first coating portion in the first region and a second coating portion in the second region, wherein the first coating portion can be arranged next to the second coating portion with respect to a main surface of the substrate and can at least partially, and preferably completely, enclose the latter.

The coating and in particular the first coating portion and/or the second coating portion can be applied to the substrate by means of any coating, vapor deposition, and/or printing method that appears appropriate to a person skilled in the art.

The coating in the second region can form an optical filter, whereby the transmission and reflection properties of the second region can be advantageously changed and/or adjusted.

With regard to the optical properties of the optical filter, reference is made to the above description of the optical properties of the optical filter element, which are equally conceivable here for the optical filter.

The illumination unit can have an optical unit that is configured to provide the illumination at the beam splitter element of the combination unit with a beam cross-section that spans the second region completely and the first region at least in portions. As a result, a high yield of illumination can be made possible.

An area of the beam cross-section of the illumination at the beam splitter element of the combination unit can be at least 200 mm², preferably at least 300 mm², and particularly preferably at least 400 mm². The area of the beam cross-section of the illumination at the beam splitter element of the combination unit can be at most 2,500 mm², preferably at most 2,250 mm², and particularly preferably at most 2,000 mm².

The optical unit can comprise a condenser lens, in particular one of the aforementioned condenser lenses, or a condenser, in particular one of the aforementioned condensers, a compensator lens, in particular one of the aforementioned compensator lenses, and/or a focus lens, in particular the aforementioned focus lens. The illumination can be collimated or substantially or approximately collimated when it hits the beam splitter element of the combination unit.

If an area of the beam cross-section is larger than a light entry area of the second region by at least a factor of 2, preferably at least a factor of 4, preferably at least a factor of 10, and particularly preferably at least a factor of 100, a particularly advantageously high light yield of the illumination can be ensured.

In some embodiments, the further illumination unit can have a further optical unit that is configured to focus the further illumination so that it passes the beam splitter element of the combination unit only within the second region. As a result, the light yield of the further illumination can be maximized. Furthermore, heating of the beam splitter element of the combination unit can be reduced or even avoided.

The light entry area of the second region can be a factor of 2 to 3 larger than a focus cross-sectional area of the further illumination in the second region.

The further optical unit can comprise a condenser lens, in particular one of the aforementioned condenser lenses, or a condenser, in particular one of the aforementioned condensers, a compensator lens, in particular one of the aforementioned compensator lenses, and/or a focus lens, in particular the aforementioned focus lens.

The illumination device can comprise a converging lens, in particular the aforementioned focus lens, which is configured to focus the combination illumination onto the optical interface. As a result, a light yield and/or homogeneity can be advantageously increased.

The illumination device can optionally have a homogenizer in front of the illumination output to homogenize the output illumination.

If objects are named in this document using number words, such as first, second, third object, etc., these are used to name and/or assign objects. Accordingly, for example, a first object and a third object may be included, but not a second object. The same applies to the designation “further.”

The devices, units, and systems disclosed herein are hereby not to be limited to the application and embodiment described above. In particular, they can have a number of individual elements, components, and units which differ from a number stated herein, in order to fulfill a function described herein. In addition, for the ranges of values specified in this disclosure, values within the stated limits shall also be deemed to be disclosed and to be usable in any manner.

The present invention is described below by way of example with reference to the accompanying figures. The drawings, the description, and the claims contain numerous features in combination. A person skilled in the art will also, expediently, consider the features individually and, in the context of the claims, use them in combination as appropriate.

If there is more than one example of a certain object, if applicable, only one of them is provided with a reference sign in the figures and in the description. The description of this example can be transferred accordingly to the other examples of the object.

### BRIEF DESCRIPTION OF THE DRAWINGS

In the drawings:

FIG. 1 shows a medical imaging device having an imaging unit in the form of an endoscope and having an illumination device connected to the endoscope,

FIG. 2 is a schematic representation of the illumination device, which has an illumination unit and a further illumination unit,

FIG. 3 is a schematic representation of a beam path of the illumination device, which has a combination unit to form a combination illumination from an illumination of the illumination unit and a further illumination of the further illumination unit,

FIG. 4 shows a beam splitter element of the combination unit, which comprises a broadband mirror having an integrating pinhole aperture, in a side sectional view,

FIG. 5 is a top view of the beam splitter element with the beam cross-section of the illumination drawn in,

FIG. 6 is a diagram having an illumination spectrum of the illumination and another illumination spectrum of the further illumination,

FIG. 7 is a diagram of a method for operating the illumination device,

FIG. 8 shows an embodiment variant of a beam splitter element that has an optical filter element in a pinhole aperture, in a side sectional view, and

FIG. 9 shows a further exemplary embodiment of a beam splitter element that has a transmitting substrate with a coating, in a side sectional view.

### DETAILED DESCRIPTION

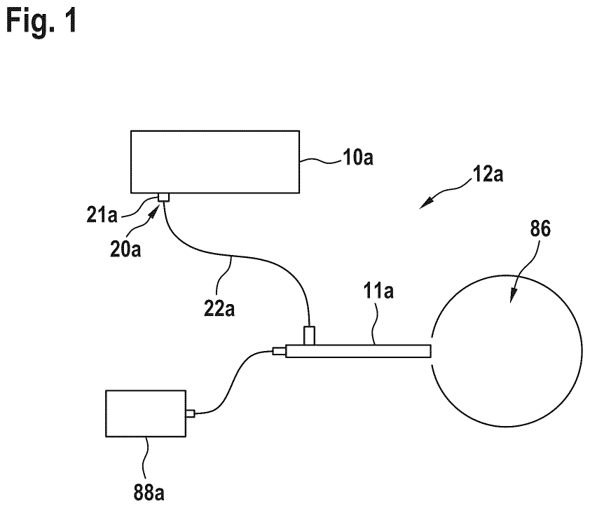

FIG. 1 shows a schematic representation of an imaging device 12a. In the exemplary case, the imaging device 12a is an endoscopic medical imaging device. Alternatively, the medical imaging device 12a could be an exoscopic, microscopic, or macroscopic medical imaging device. The imaging device 12a is configured, for example, for examining a cavity 86a.

The imaging device 12a has a medical imaging unit 11a. In the illustrated case, this is an endoscope that can be partially inserted into cavity 86a.

The imaging device 12a comprises an illumination device 10a having an optical interface 21a. The imaging unit 11a can be optically connected to the optical interface 21a. The optical interface 21a can be part of an optical-mechanical interface that can be optionally detachable and connectable. The imaging unit 11a can optionally be decoupled from the illumination device 10a.

The illumination device 10a is configured to provide output illumination at an illumination output 20a that has the optical interface 21a. The output illumination can be fed to the imaging unit 11a by means of a light guide 22a of the imaging device 12a. The imaging unit 11a couples the output illumination to an object to be imaged, such as a situs.

In the illustrated case, the imaging device 12a further comprises a display unit 88a on which images can be displayed that are based upon image data acquired by the imaging unit 11a. These can be video images, still images, overlays of different images, partial images, image sequences, etc.

The imaging device 12a is multimodal. By way of example, the imaging device can be operated in three basic modes: a multispectral mode, a fluorescence mode, and a white-light mode. It can further be provided for the imaging device 12a to be operable in a hyperspectral mode in addition or as an alternative to the multispectral mode. The illumination device 10a is multimodal. The illuminating device 10a is operable in different illumination modes, in which it provides light for different imaging modes. In the present case, the illumination device 10a is operable in three basic modes, viz., a multispectral mode, a fluorescence mode, and a white-light mode. The imaging unit 11a is also operable in different operating modes, specifically also in at least a multispectral mode, a fluorescence mode, and a white-light mode. In the corresponding operating mode of the imaging device 12a, the modes of the illumination device 10a and/or the imaging unit 11a are matched to one another.

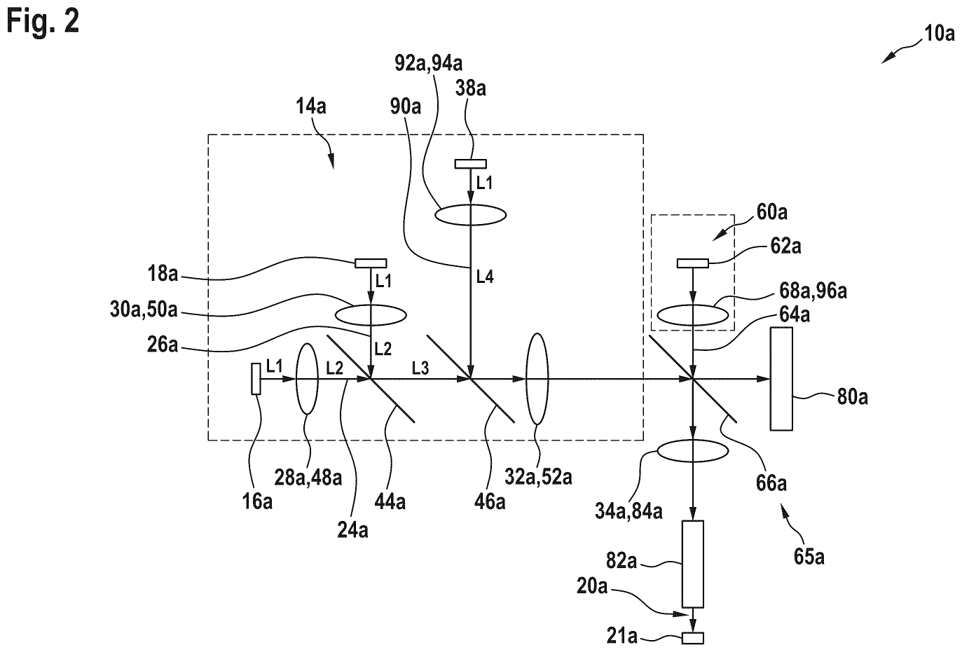

FIG. 2 is a schematic representation of the illumination device 10a. The illumination device 10a comprises an illumination unit 14a that is configured to provide illumination. The illumination device 10a has a further illumination unit 60a that is configured to provide further illumination.

The illumination device 10a has a combination unit 65a with a beam splitter element 66a, which is configured to combine a portion of the illumination and a portion of the further illumination to form a combination illumination, which is then provided as output illumination.

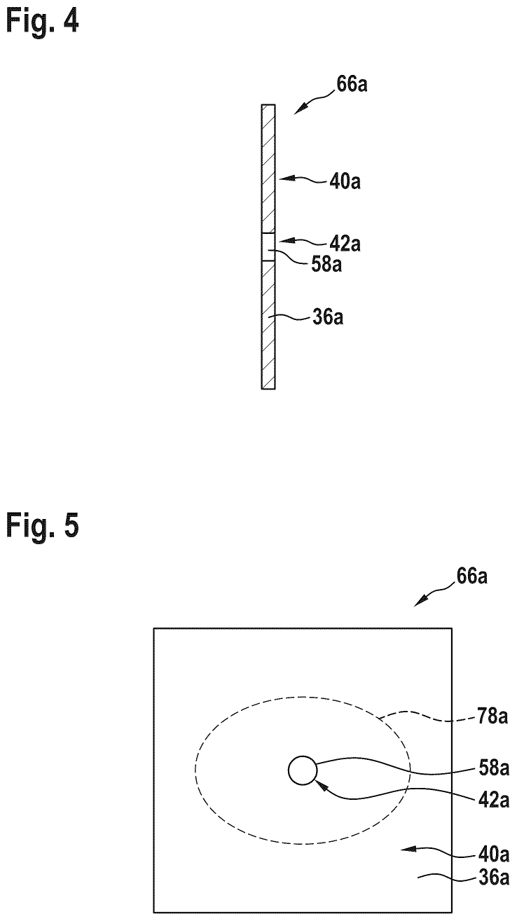

The beam splitter element 66a is shown in FIG. 4 in a side sectional view and in FIG. 5 in a top view. The beam splitter element 66a has a first region 40a that is substantially reflective for the illumination and a second region 42a that is substantially transmissive for the further illumination and that is completely surrounded by the first region 40a. The beam splitter element 66a has a flat broadband mirror 36a having an integrating pinhole aperture 58a, which defines the second region 42a. The pinhole aperture 58a is formed by a vertical through-hole in the broadband mirror 36a.

By means of the beam splitter element 66a, the illumination and the further illumination are combined in that the illumination is reflected at the broadband mirror 36a, while the further illumination is transmitted through the pinhole aperture 58a. The further illumination is focused in the pinhole aperture 58a. By means of the beam splitter element 66a, an illumination spectrum 54 of the illumination and a further illumination spectrum 56 of the further illumination are in each case combined substantially unchanged.

The illumination device 10a has a focus lens 84a behind the beam splitter element 66a of the combination unit 65a in the direction of the illumination output 20a (cf. FIG. 2). The output illumination supplied to the illumination output 20a is focused by means of the focus lens 84a. The illumination device 10a can additionally have a homogenizer 82a, e.g., in the form of a homogenizer rod, for homogenizing the output illumination upstream of the illumination output 20a, but this is not required.

The illumination unit 14a has a first illumination source 16a, a second illumination source 18a, and a third illumination source 38a. Alternatively, the illumination unit 14a could also have more or fewer illumination sources 16a, 18a, 38a.

The first illumination source 16a is designed as an LED. The first illumination source 16a is configured to emit narrow-band light at a mean wavelength of 940 nm.

The second illumination source 18a is designed as an LED. The second illumination source 18a is configured to emit narrow-band light at a mean wavelength of 660 nm.

The third illumination source 38a is designed as an LED. The third illumination source 38a is configured to emit broadband light having intensity maxima at 440 nm and 550 nm.

The further illumination unit 60a has a further illumination source 62a. Alternatively, the further illumination unit 60a could also have several further illumination sources 62a, the radiation of which can be combined by means of beam splitters and/or fiber combiners of the further illumination unit 60a.

The further illumination source 62a is designed as a laser diode. The further illumination source 62a is configured to emit a laser line at a mean wavelength of 470 nm, 640 nm, or 770 nm. In the case of several further illumination sources 62a, the further illumination unit 60a could have a separate further illumination source 62a for each of the wavelengths mentioned. The illumination sources 16a, 18a, 38a and the further illumination source 62a can be activated independently of one another or in groups to realize the different operating modes.

In alternative embodiments, other mean wavelengths and/or bandwidths and/or spectra for the illumination sources 16a, 18a, 38a and the further illumination source 62a would also be conceivable.

The illumination device 10a has, for each illumination source 16a, 18a, 38a of the illumination unit 14a, an optical path 24a, 26a, 90a, which extends from the particular illumination source 16a, 18a, 38a to the illumination output 20a.

A first optical path 24a extends from the first illumination source 16a to the illumination output 20a and comprises exactly three optical beam-shaping elements 28a, 32a, 34a.

A second optical path 26a extends from the second illumination source 18a to the illumination output 20a and comprises exactly three optical beam-shaping elements 30a, 32a, 34a.

A third optical path 90a extends from the third illumination source 38a to the illumination output 20a and comprises exactly three optical beam-shaping elements 32a, 34a, 92a.

Thus, all optical paths 24a, 26a, 90a from the illumination sources 16a, 18a, 38a of the illumination unit 14a to the illumination output 20a in each case comprise exactly three optical beam-shaping elements 28a, 30a, 32a, 34a, 92a.

The optical beam-shaping elements 28a, 30a, 92a are designed as condenser lenses 48a, 50a, 94a, specifically as a first condenser lens 48a spaced by a distance L1 from the first illumination source 16a, a second condenser lens 50a spaced by a distance L1 from the second illumination source 18a, and a third condenser lens 94a spaced by a distance L1 from the third illumination source 38a. The first condenser lens 48a, the second condenser lens 50a, and the third condenser lens 94a are identical to one another.

The optical beam-shaping element 32a is formed as a common compensator lens 52a, which is common to the first optical path 24a, the second optical path 26a, and the fourth optical path 90a.

The optical beam-shaping element 34a is designed as the focus lens 84a and is common to all optical paths 24a, 26a, 90a.

The illumination device 10a has two beam splitter elements 44a, 46a, each of which is configured to couple one of the illumination sources 18a, 38a of the illumination unit 14a into a beam path starting from the illumination source 16a in the direction of the illumination output 20a.

The beam splitter element 44a is part of the first optical path 24a and the second optical path 26a. In each case, the beam splitter element 44a is spaced by a distance L2 from the first condenser lens 48a and the second condenser lens 50a. The beam splitter element 44a has an edge filter having an edge that lies in the wavelength space between a wavelength range emitted by the first illumination source 16a and a wavelength range emitted by the second illumination source 18a.

The beam splitter element 46a is part of the first optical path 24a, the second optical path 26a, and the third optical path 90a. The beam splitter element 46a is spaced by a distance L4 from the third condenser lens 94a. The beam splitter element 46a has an edge filter having an edge that lies in the wavelength space between a wavelength range emitted by the first illumination source 16a and the second illumination source 18a, and a wavelength range emitted by the third illumination source 38a.

The beam splitter elements 44a, 46a are arranged optically directly one behind the other and spaced by a distance L3. The common compensator lens 52a is arranged optically between the beam splitter element 46a and the beam splitter element 66a of the combination unit 65a.

The beam splitter element 66a of the combination unit 65a and, if applicable, the homogenizer 82a are part of the first optical path 24a, the second optical path 26a, and the third optical path 90a.

The first optical path 24a, the second optical path 26a, and the third optical path 90a are of equal length, since the following relationship applies between the distances L2, L3, and L4:

L4=L2+L3

The first optical path 24a, the second optical path 26a, and the third optical path 90a also have the same optical beam-shaping elements 28a, 30a, 32a, 34a, 92a. The first optical path 24a, the second optical path 26a, and the third optical path 90a are thus identical to one another.

For each of the illumination sources 16a, 18a, 38a of the illumination unit 14a, a fundamentally similar beam path to the illumination output 20a results: The beam path for each of the illumination sources 16a, 18a, 38a always runs via the condenser lens 48a, 50a, 94a assigned to it, via the common compensator lens 52a and via the focus lens 84a.

The illumination device 10a has a further optical path 64a for each further illumination source 62a of the further illumination unit 60a—in the present case, only one—which extends from the particular further illumination source 62a to the illumination output 20a.

The further optical path 64a extends from the further illumination source 62a to the illumination output 20a and comprises exactly two optical beam-shaping elements 34a, 68a.

The optical beam-shaping element 68a is designed as a condenser lens 96a, specifically as a further condenser lens 96a assigned to the further illumination source 62a. The further condenser lens 96a differs from the first condenser lens 48a, the second condenser lens 50a, and the third condenser lens 94a, but could also be identical in alternative embodiments.

The optical beam-shaping element 34a is in turn designed as the focus lens 84 and is common to the optical paths 24a, 26a, 90a.

Since the further illumination source 62a is designed as a strong laser diode, and the further optical path 64a is relatively short, a compensator lens can be omitted in the further optical path 64a. The radiation losses are manageable here.

The further optical path 64a also comprises the beam splitter element 66a of the combination unit 65a and, if applicable, the homogenizer 82a.

The illumination unit 14a has an optical unit 76a that is configured to provide the illumination at the beam splitter element 66a with a beam cross-section 78a that completely spans the second region 42a and at least partially spans the first region 40a. This is not shown to scale in FIG. 5. An area of the beam cross-section 78a is at least a factor of 100 larger than a light entry area of the second region 42a. The optical unit 76a comprises the first condenser lens 48a, the second condenser lens 50a, the third condenser lens 94a, and the common compensator lens 52a.

The further illumination unit 60a has a further optical unit 98a that is configured to focus the further illumination so that it passes the beam splitter element 66a only within the second region 42. The further optical unit 98a comprises the further condenser lens 96a.

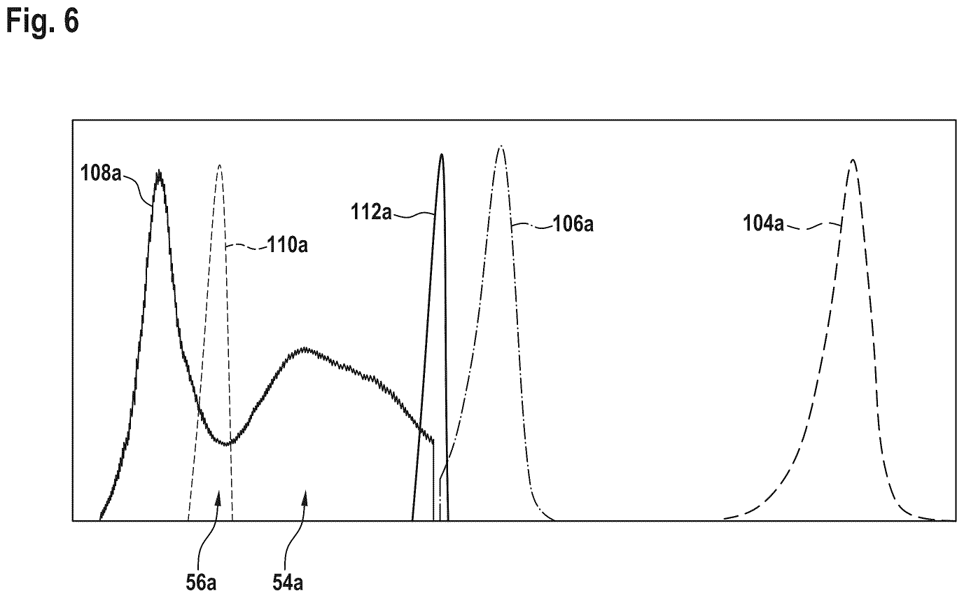

FIG. 6 shows a diagram with the illumination spectrum 54a and the further illumination spectrum 56a.

The illumination spectrum 54a comprises a first individual illumination spectrum 104a, which originates from the first illumination source 16a. The illumination spectrum 54a comprises a second individual illumination spectrum 106a, which originates from the second illumination source 18a. The illumination spectrum 54a comprises a third individual illumination spectrum 108a, which originates from the third illumination source 38a. The sharp cuts in the illumination spectrum 54a between the second individual illumination spectrum 106a and the third individual illumination spectrum 108a are due to the beam splitter element 46a.

The further illumination spectrum 56a comprises a further first individual illumination spectrum 110a, which originates from the further illumination source 62a. In FIG. 6, the case of a further illumination unit 60a with two further illumination sources 62a is also shown by way of example. Then, the further illumination spectrum 56a could have a further second individual illumination spectrum 112a.

The illumination spectrum 54a is designed to be broader-band than the further illumination spectrum 56a. In particular, the individual illumination spectra 104a, 106a, 108a are in each case broader-band than the other individual illumination spectra 110a, 112a. In addition, the illumination spectrum 54a overlaps at least partially in the wavelength space with the further illumination spectrum 56a. In particular, the third individual illumination spectrum 108a overlaps with the further first individual illumination spectrum 110a and the further second individual illumination spectrum 112a. Furthermore, the second individual illumination spectrum 106a overlaps with the further second individual illumination spectrum 112a.

Returning to FIG. 2: The illumination device 10a comprises a detector unit 80a to determine at least one illumination property of the output illumination, specifically the illumination provided by the illumination unit 14a. A portion of the illumination passes through the pinhole aperture 58a onto the detector 80a and can thus be used to monitor the illumination unit 14a by means of the detector 80a.

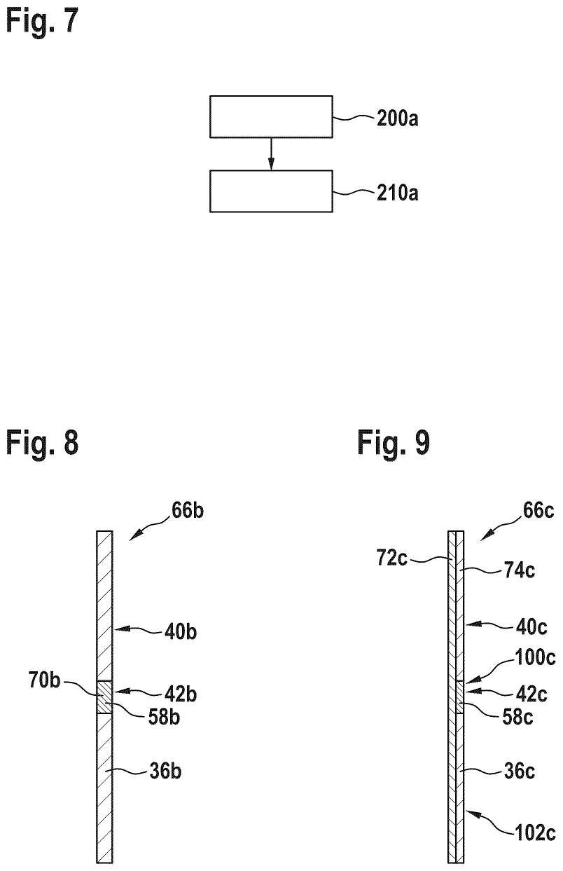

FIG. 7 shows a schematic flowchart of a method for operating the illumination device 10a. In a step 200a, the illumination unit 14a and the further illumination unit 60a are activated. Depending upon requirements, certain of the illumination sources 16a, 18a, 38a and further illumination sources 62a are activated. If both one of the illumination sources 16a, 18a, 38a of the illumination unit 14a and the further illumination source 62a are activated, in a step 210a, at least a portion of the illumination and at least a portion of the further illumination are combined to form a combination illumination. The portion of the illumination and the portion of the further illumination are combined to form the combination illumination by means of the beam splitter element 66a. The illumination spectrum 54a and the further illumination spectrum 46a are in each case combined substantially unchanged.

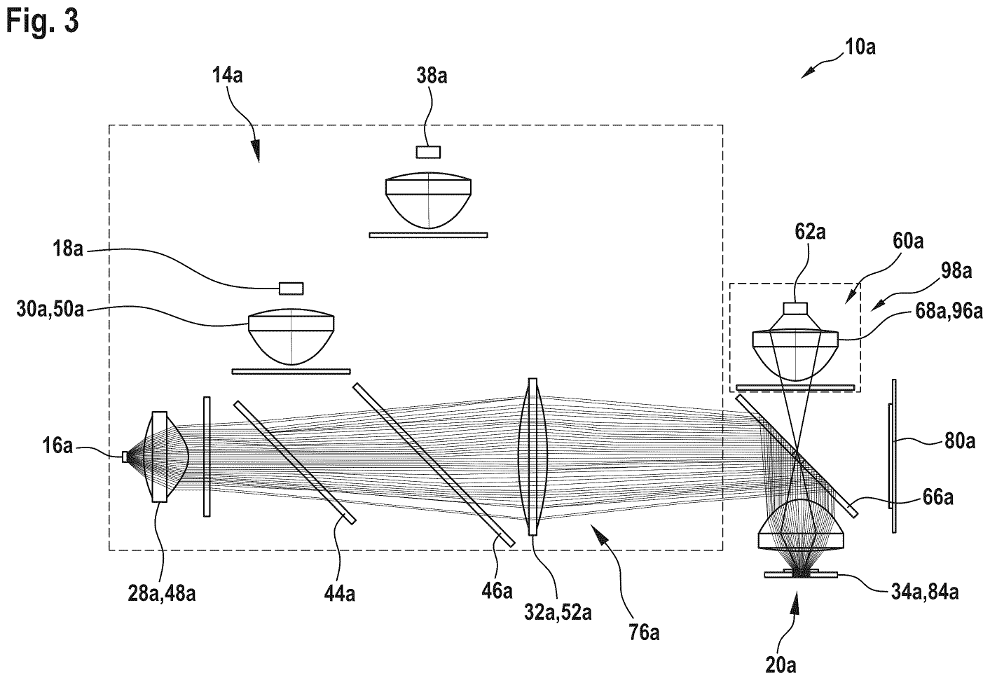

Further exemplary embodiments of the invention are shown in FIGS. 8 and 9. The following description is substantially limited to the differences between the exemplary embodiments. With regard to structural units and components with the same reference signs, reference can generally be made to the description of the other exemplary embodiments, in particular the exemplary embodiment of FIGS. 1 to 7. For differentiation, the reference signs of the exemplary embodiments of FIGS. 8 and 9 are followed by one of the letters “b” or “c” instead of the letter “a” of the exemplary embodiment of FIGS. 1 to 7.

The exemplary embodiment shown in FIG. 8 differs from the previous exemplary embodiment in that a beam splitter element 66b in a second region 42b has an optical filter element 70b. This optical filter element 70b is precisely fitted and, in particular, glued into a pinhole aperture 58b of a broadband mirror 36b of the beam splitter element 66b.

Over an entire wavelength range of the illumination and the further illumination, the optical filter element 70b reflects a small proportion of at most 1% of an incident light intensity, and transmits the rest.

Thus, with reference to the previous exemplary embodiment, the illumination unit 14a and the further illumination unit 60a can be monitored by means of the detector unit 80a, specifically both jointly, in a joint operation of the illumination unit 14a and the further illumination unit 60a, and individually, in the case of sequential activation of the illumination unit 14a and the further illumination unit 60a one after the other.

In the exemplary embodiment shown in FIG. 9, a beam splitter element 66c has a substrate 72c that is substantially transmissive for illumination and further illumination, having a coating 74c that reflects broadband in a first region 40c and, in a second region 42c, is designed to be partially reflective for illumination and partially transmissive for further illumination.

The coating 74c has a first coating portion 100c in the first region 40c and a second coating portion 102c in the second region 42c. The first coating portion 100c is arranged next to the second coating portion 102c with respect to a main surface of the substrate 72c and completely encloses the latter. The second coating portion 102c is designed in the shape of a circular disk.

The coating 74c forms an optical filter in the second region 42c. With regard to filter properties of the filter, reference can be made to the filter properties of the optical filter element 70b from the previous exemplary embodiment.

### LIST OF REFERENCE SIGNS

10 Illumination device
11 Imaging unit
12 Imaging device
14 Illumination unit
16 First illumination source
18 Second illumination source
20 Illumination output
21 Optical interface
22 Light guide
24 First optical path
26 Second optical path
28 Optical beam-shaping element
30 Optical beam-shaping element
32 Optical beam-shaping element
34 Optical beam-shaping element
36 Broadband mirror
38 Third illumination source
40 First region
42 Second region
44 Beam splitter element
46 Beam splitter element
48 First condenser lens
50 Second condenser lens
52 Common compensator lens
54 Illumination spectrum
56 Further illumination spectrum
58 Pinhole aperture
60 Further illumination unit
62 Further illumination source
64 Further optical path
65 Combination unit
66 Beam splitter element
68 Optical beam-shaping element
70 Optical filter element
72 Substrate
74 Coating
76 Optical unit
78 Beam cross-section
80 Detector unit
82 Homogenizer
84 Focus lens
86 Cavity
88 Display unit
90 Third optical path
92 Optical beam-shaping element
94 Third condenser lens
96 Further condenser lens
98 Further optical unit
100 First coating portion
102 Second coating portion
104 First individual illumination spectrum
106 Second individual illumination spectrum
108 Third individual illumination spectrum
110 Further first individual illumination spectrum
112 Further second individual illumination spectrum
200 Step
210 Step

1. An illumination device for a medical imaging unit such as an endoscope, exoscope, and/or microscope, comprising:
an illumination unit that is configured to provide illumination with an illumination spectrum,
a further illumination unit that is configured to provide further illumination with a further illumination spectrum, and
a combination unit that is configured to combine at least a portion of the illumination and at least a portion of the further illumination to form a combination illumination,
wherein the combination unit has a beam splitter element that has a first region that is substantially reflective for the illumination and a second region that is substantially transmissive for the further illumination, which second region is arranged next to the first region and is at least partially surrounded by the first region.

2. The illumination device according to claim 1,
wherein the illumination spectrum in the wavelength space at least partially overlaps with the further illumination spectrum.

3. The illumination device according to claim 1,
wherein the illumination spectrum is broader-band than the further illumination spectrum.

4. The illumination device according to claim 1,
wherein at least one illumination source of the illumination unit comprises an LED.

5. The illumination device according to claim 1,
wherein at least one further illumination source of the further illumination unit comprises a laser or a laser diode.

6. The illumination device according to claim 1,
wherein the second region is at least partially transmissive for the illumination.

7. The illumination device according to claim 1,
wherein the second region is at least partially reflective for further illumination.

8. The illumination device according to claim 1,
wherein the beam splitter element of the combination unit comprises a broadband mirror having an integrating pinhole aperture, which defines the second region.

9. The illumination device according to claim 8,
wherein the beam splitter element of the combination unit has an optical filter element in the second region.

10. The illumination device according to claim 1,
wherein the beam splitter element of the combination unit has a substrate that is substantially transmissive for the illumination and the further illumination, and a coating on the substrate that reflects broadband in the first region and that is at least partially transmissive in the second region for the further illumination.

11. The illumination device according to claim 10,
wherein the coating in the second region forms an optical filter.

12. The illumination device according to claim 1,
wherein the illumination unit has an optical unit that is configured to provide the illumination at the beam splitter element of the combination unit with a beam cross-section that spans the second region completely and spans the first region at least in portions.

13. The illumination device according to claim 12,
wherein an area of the beam cross-section is at least a factor of 2 larger than a light entry area of the second region.

14. The illumination device according to claim 1,
wherein the further illumination unit has a further optical unit that is configured to focus the further illumination so that it passes the beam splitter element of the combination unit only within the second region.

15. The illumination device according to claim 1,
wherein an illumination output that has an optical interface and is configured to provide an output illumination that is at least partially formed by the combination illumination, and
a focus lens that is configured to focus the combination illumination onto the optical interface.

16. The illumination device according to claim 1,
wherein the illumination spectrum in the wavelength space at least partially overlaps with the further illumination spectrum,
wherein the combination unit is configured to combine the illumination spectrum and the further illumination spectrum in each case substantially unchanged.

17. The illumination device according to claim 1,
wherein the illumination device comprises a detector unit for determining at least one illumination property of the output illumination and/or the combination illumination.

18. The medical imaging unit, having an illumination device according to claim 1.

19. A method for operating an illumination device comprising:
providing an illumination unit that is configured to provide illumination with an illumination spectrum,
providing a further illumination unit that is configured to provide further illumination with a further illumination spectrum,
combining at least a portion of the illumination and at least a portion of the further illumination to form a combination illumination,
wherein combining the portion of the illumination and the portion of the further illumination to form the combination illumination by a beam splitter element that has a first region that is substantially reflective for the illumination and a second region that is substantially transmissive for the further illumination, which second region is arranged next to the first region and is at least partially surrounded by the first region.

20. The method according to claim 19,
wherein the illumination spectrum in the wavelength space at least partially overlaps with the further illumination spectrum,
wherein the illumination spectrum and the further illumination spectrum are in each case combined substantially unchanged.
